(19) United States
(12) Patent Application Publication
(10) Pub. No.: US 2012/0141690 A1
(43) Pub. Date: Jun. 7, 2012

(54) PROCESS FOR PERMANENT DEFORMATION OF KERATIN FIBERS

(75) Inventors: Hiroshi Takahashi, Osaka (JP); Maxime De Boni, Tokyo (JP)

(73) Assignee: L'OREAL, PAris (FR)

(21) Appl. No.: 13/382,369

(22) PCT Filed: Jul. 10, 2009
(86) PCT No.: PCT/JP09/62936

§ 371 Date: Feb. 24, 2012

(51) Int. Cl. B05D 3/06; C01C 1/26; H05B 6/02; C01D 7/00; B05D 7/18; C07C 69/96

(52) U.S. Cl. 427/544; 427/222; 427/557; 427/554; 558/260; 118/642; 558/262; 423/422; 423/420; 423/421

(57) ABSTRACT

The present invention relates to a process for permanent deformation of keratin fibers comprising the steps of : providing the keratin fibers with mechanical fibers a composition comprising one or several sources of ions of formula: wherein X is a group selected from the group consisting of O⁻, OH, NH₂, O—OH, and O—COO⁻; then placing the keratin fibers in an occlusive space; and then heating the keratin fibers. The present invention also relates to an agent and a kit to be used for the above process.

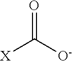

### TECHNICAL FIELD

The present invention relates to a process for the permanent deformation of keratin fibers such as hair, as well as an agent and a kit to be used for the process.

### BACKGROUND ART

Many hair care products are marketed nowadays to easily style, texturize and add some weight to the hair, and especially to thin hair, amongst which foams and styling gels or hair lacquers may be mentioned as an example. These products enable shaping of the hair but are removed with shampoo and thus are required to be applied on a daily basis.

The most general technique for obtaining a long-lasting deformation of the hair consists, in a first step, of opening the keratin S—S disulfide bonds (cystine) with a composition comprising a suitable reducing agent (reducing step) then, once the thus treated hair has been rinsed, generally with water, reforming said disulfide bonds in a second step, by applying an oxidizing composition onto the hair which has been placed beforehand under tension, using curlers for example (oxidizing step, also called fixing step) so as to give the hair the desired form in the end.

The new shape that is imposed to the hair by means of a chemical treatment, such as explained above, is relatively long-lasting and is particularly resistant to washing operations with water or shampoo, as compared to the usual simple methods for temporarily reshaping the hair by using foams, styling gels, or lacquers.

Many compositions and processes for the above chemical treatment have been proposed. Generally, they offer good performances on the day of treatment.

### DISCLOSURE OF INVENTION

However, there are various drawbacks as follows in the above chemical treatment process that may not be suitable from the view point of consumer's expectations:
Insufficient long-lastingness against environmental stress (mechanical constraints from brushing, frequent shampoos, light exposure, high humidity and the like);
Insufficient perm efficiency on natural hair;
High levels of hair degradation, especially in repeated applications or in combination with other chemical treatments such as oxidative coloration;
Long processing time; and
Malodor of thiol-compounds during and after the perm process.

Thus, an objective of the present invention is to provide a new permanent deformation process for keratin fibers such as hair, which provides good curl lastingness.

Another objective of the present invention is to provide strong perm efficiency for natural hair by the above new permanent deformation process for keratin fibers.

Another objective of the present invention is to prevent high level of hair damage by a permanent deformation process for keratin fibers.

A further objective of the present invention is to reduce the time required for conventional permanent deformation processes for keratin fibers, providing a quick permanent shaping process for keratin fibers.

A further objective of the present invention is to prevent malodor which is derived from thiol-compounds which are commonly used in conventional permanent deformation processes for keratin fibers.

The above objectives of the present invention can be achieved by a process for permanent deformation of keratin fibers comprising the steps of:
providing the keratin fibers with mechanical tension for the deformation;
applying onto the keratin fibers a composition comprising one or several sources of ions of formula:

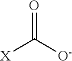

wherein
X is a group selected from the group consisting of O⁻, OH, NH₂, O—OH, and O—COO⁻;
then placing the keratin fibers in an occlusive space; and
then heating the keratin fibers.

The above process may further comprise the step of rinsing the keratin fibers after the step of applying the composition onto the keratin fibers and/or after the step of heating the keratin fibers.

The occlusive space may be formed by at least one coating means. The coating means may be rigid or flexible. The coating means may comprise at least one member selected from the group consisting of a film and a sheet.

The mechanical tension may be provided by at least one reshaping means selected from the group consisting of a curler, a roller, a plate and an iron.

In the above process, the keratin fibers may be heated at 50° C. to 250° C. during the step of heating the keratin fibers. The keratin fibers may be heated by at least one heater providing at least one selected from the group consisting of hot air, hot steam, high frequency induction heating, microwave heating, infra-red ray irradiation, laser, and flash lamp irradiation. The coating means and/or the reshaping means may comprise the heater.

The composition used in the above process may comprise the sources of ions of the invention in a total amount of 0.1 to 40% by weight relative to the total weight of the composition. The pH of the composition may range from 6 to 11. The ions may be selected from the group consisting of carbonate salts, hydrogencarbonate salts, carbamate salts, peroxycarbonate salts.

The above process may comprise no step of oxidizing the keratin fibers.

Another aspect. of the present invention is an agent for one-step permanent deformation of keratin fibers to be heated in an occlusive space, comprising one or several sources of ions of formula:

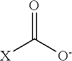

wherein

X is a group selected from the group consisting of O⁻, OH, NH₂, O—OH, and O—COO⁻.

The present invention also relates to a kit for permanent deformation of keratin fibers, comprising:

a device comprising
at least one reshaping means to provide the keratin fibers with mechanical tension,
at least one coating means to form an occlusive space, and
at least one heater to heat the keratin fibers in the occlusive space;
and

a composition comprising one or several sources of ions of formula:

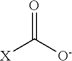

wherein
X is a group selected from the group consisting of O⁻, OH, NH₂, O—OH, and O—COO⁻.

### BEST MODE FOR CARRYING OUT OF THE INVENTION

In order to achieve good performance, conventional perm products for keratin fibers such as hair contain a relatively high concentration of a reducing agent or agents, and have high alkalinity. For example, thioglycolic acid, one of the common reducing agents in the art, is used typically in a concentration of between 6 and 1 wt % at a pH which may be 11 or more.

Additionally, some technologies use a heating process during the reducing step of keratin fibers in order to increase the chemical action of such reducing agent(s). These technologies use hot-air/hot-steam/far-infrared to heat the keratin fibers rolled up on, for example, a plastic roller. Because the heating process is performed in an open environment, the keratin fibers are heated at most less to than 50° C., and cannot be heated furthermore due to the vaporization of water or moisture in the keratin fibers. At the end of the heating step, the reduced keratin fibers are rinsed, oxidized and rinsed again. The advantage of employing this heating process is a small improvement in perm performance and curl lastingness in comparison with a classical cold perm process.

However, in order to achieve better performance, strong reducing agent(s) in a higher concentration as well as strong alkali conditions are necessary. Therefore, keratin fibers undergo a significant or critical degradation that is not preferable for customers when considering repeated applications or further chemical treatments (for example, perm and coloration).

After diligent research, the inventors have discovered that it is possible to achieve better perm performance without generating significant or critical degradation of keratin fibers by using a composition comprising a source of specific ions in association with a specific heating process during the permanent deformation treatment for the keratin fibers.

The above specific heating process is performed in a closed or occlusive environment, which limits the evaporation of water or moisture from the keratin fibers and maintains the keratin fibers at higher temperature in the wet state. Accordingly, the treated keratin fibers show good curl lastingness with good perm efficiency.

The composition used in the present invention does not need to contain any reducing agents such as thiol-compounds. Therefore, malodor derived from the reducing agents can be prevented.

Furthermore, degradation of the keratin fibers can be prevented, because no strong alkali is necessary.

According to the present invention, an oxidative step is no longer required to achieve a stable permanent wave formation, unlike in a classical perm process. Thus, a one-step process can be achieved by a combination of the above new composition with the above new heating process. This can considerably reduce the time required for permanent deformation process for keratin fibers.

(Composition)

The composition used for the present invention comprises one or several sources of ions of formula:

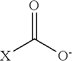

wherein

X is a group selected from the group consisting of O⁻, OH, NH₂, O—OH, and O—COO⁻.

Suitable sources of these ions may be inorganic salts such as sodium, potassium, lithium, calcium, magnesium, barium, and ammonium salts of carbonate, peroxycarbonate, carbamate, hydrogencarbonate ions and mixtures thereof. As examples of the above inorganic salts, mention may be made of sodium carbonate, sodium hydrogencarbonate, potassium carbonate, potassium hydrogencarbonate, lithium carbonate, lithium hydrogencarbonate, calcium carbonate, magnesium carbonate, barium carbonate, ammonium carbonate, ammonium hydrogencarbonate and mixtures thereof.

Suitable sources of these ions may also be organic salts such as organic base salts of carbonate, peroxycarbonate, carbamate, hydrogencarbonate ions and mixtures thereof. As an example of the above organic salts, mention may be made of amino acid carbonates and guanidine carbonate.

Among carbonate salts, ammonium carbonate, sodium carbonate, potassium carbonate, guanidine carbonate and its derivatives are preferable.

Among hydrogencarbonate (bicarbonate) salts, sodium bicarbonate, potassium bicarbonate, ammonium bicarbonate and its derivatives such as tetraethylammonium bicarbonate and triethylammonium bicarbonate are preferable.

Among carbamate salts, ammonium carbamate is preferable.

Among peroxycarbonate salts, sodium peroxycarbonate and potassium peroxycarbonate are preferable.

As the ion sources, carbonate salts and hydrogencarbonate salts are preferable. In particular, sodium carbonate and sodium hydrogencarbonate (called bicarbonate) are more preferable.

The sources of the above ions may be used in a total amount of from 0.1 to 40% by weight, preferably from 0.2 to 30% by weight, more preferably from 0.3 to 20% by weight, relative to the total weight of the composition, depending on their solubility.

The pH of the composition may range from 6 to 11, preferably between 7.5 and 9.5, and more preferably between 8.0 to 9.0. Since the pH of the composition is not relatively high, damage to the keratin fibers by the composition can be reduced.

In order to adjust the pH, an acidic or alkali agent(s) other than sources of ions of the invention may be used alone or in combination. The amount of the acidic or alkali agent(s) is not limited, but may be from 0.1 to 5% by weight relative to the total weight of the composition. As the acidic agents, mention may be made of any inorganic or organic acids which are commonly used in cosmetic products such as citric acid, lactic acid, phosphoric acid or hydrochloric acid (HC1). HCl is preferable. As the alkali agents, mention may be made of any inorganic or organic basic agents which are commonly used in cosmetic products such as ammonia; alcanolamines such as mono-, di- and tri-ethanolamine, isopropanolamine; sodium and potassium hydroxides; urea, guanidine and their derivatives; basic amino acids such as lysine or arginine; and diamines such as those described in the structure below:

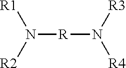

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine and monoethanolamine are preferred.

The composition used in the present invention may also comprise one or more cosmetic agent(s). The amount of the cosmetic agent(s) is not limited, but may be from 0.1 to 10% by weight relative to the total weight of the composition. The cosmetic agent(s) may be selected from the group consisting of volatile or non volatile, linear or cyclic, amine-type or not, silicones, cationic, anionic, non ionic or amphoteric polymers, peptides and derivatives thereof, protein hydrolyzates, synthetic or natural waxes, and especially fatty alcohols, swelling agents and penetrating agents, as well as other active compounds, such as anionic, cationic, non ionic, amphoteric or zwitterionic surfactants, agents for combating hair loss, anti-dandruff agents, associative-type or not, natural or synthetic thickeners, suspending agents, sequestering agents, opacifying agents, dyes, sunscreen agents, fillers, vitamins or provitamins, mineral, vegetable or synthetic oils, as well as fragrances, preserving agents, stabilizers, reducing agents and mixtures thereof.

The vehicle for the composition used in the present invention is preferably an aqueous medium consisting of water and may advantageously contain one or several cosmetically acceptable organic solvents, which particularly include alcohols, such as ethyl alcohol, isopropyl alcohol, benzyl alcohol and phenylethyl alcohol, or polyols or polyol ethers, such as ethylene glycol monomethyl, monoethyl and monobutyl ethers, propylene glycol or ethers thereof, such as propylene glycol monomethylether, butylene glycol, dipropylene glycol as well as diethylene glycol alkyl ethers, such as diethylene glycol monoethylether or monobutylether. The water may be present in a concentration of from 10 to 90% by weight relative to the total weight of the composition. The organic solvent(s) may then be present in a concentration of from 0.1 to 20% by weight, and preferably from 1 to 10% by weight elative to the total weight of the composition.

The composition may contain conventional reducing agents such as thioglycolic acid which is used in a conventional hair care product used for permanent deformation of keratin fibers. Preferably, the composition does not contain this kind of products in order to avoid bad odor.

The composition used in the present invention may exist in any form such as a lotion, a gel, thickened or not, a foam, or a cream.

(Heating Process)

According to the present invention, keratin fibers such as hair are subjected to a specific heating process which is performed in an occlusive space.

The heating process may preferably be performed by using a special heating device or devices that can form an occlusive space to restrict the evaporation of evaporable components such as water in the above-described composition from keratin fibers and keep a predetermined temperature in the heating device throughout the process.

If the evaporable components such as water in the above-described composition evaporate from keratin fibers, most of the heat energy applied to the keratin fibers will be consumed by the evaporation, and therefore the temperature of the keratin fibers cannot increase up to the predetermined temperature until all evaporable components in the composition evaporate.

The above heating device may comprise a heat energy source being either in contact with keratin fibers or apart from keratin fibers; and at least one means to form an occlusive space surrounding the keratin fibers.

The heat energy source is used to heat keratin fibers. The heat energy source may be at least one heater providing at least one selected from the group consisting of hot air, hot steam, high frequency induction heating, microwave heating, infra-red ray irradiation, laser, and flash lamp irradiation.

The occlusive space may be formed by at least one coating means. A plurality of coating means may be used. The coating means may be rigid or flexible.

The coating means may comprise at least one member selected from the group consisting of a film and a sheet. The material of the film or the sheet is not limited. For example, the film or the sheet may comprise a thermoplastic or thermosetting resin, a paper, a textile, a metal foil such as aluminum foil, and the like.

For example, the film or sheet may be set on a heating rod, a heating bar or a heating plate which is covered by keratin fibers.

According to the present invention, the coating means may comprise the heat energy source. Therefore, for example, the film or sheet which includes a heater may be set on a rod, a bar, or a plate which is covered by keratin fibers.

The occlusive conditions can restrict the evaporation of evaporable components such as water in the above-described composition applied to keratin fibers, and therefore the temperature of the keratin fibers can be increased higher than that obtainable by a conventional heating process or device for the keratin fibers in open conditions. Accordingly, the keratin fibers can be reshaped effectively, and the shape given to the keratin fibers can last for a long time.

The keratin fibers can be heated at 50° C. to 250° C., preferably 60° C. to 200° C., more preferably 60° C. to 150° C., more preferably 60° C. to 90° C., during the step of heating the keratin fibers.

The heating process may be performed for an appropriate time which is required to deform the shape of keratin fibers. The time length for the heating process is not limited, but it may be from 1 minute to 2 hours, preferably 1 minute to 1 hour, and more preferably 1 minute to 30 minutes.

(Permanent Deformation Process for Keratin Fiber)

The permanent deformation process for keratin fibers according to the present invention may be performed as follows.

First, keratin fibers are subjected to mechanical tension for deformation. The mechanical tension can be applied to the keratin fibers by any means to deform the keratin fibers to an intended shape. For example, the mechanical tension may be provided by at least one reshaping means selected from the group consisting of a curler, a roller, a plate and an iron. The reshaping means may comprise at least one heater as described above.

Next, the above-described composition is applied to the keratin fibers. The application of the composition may be performed by any means, such as a brush and a comb. The keratin fibers to which the mechanical tension has been applied should be treated with the composition.

Lastly, the above-described heating process is performed. The heat energy is applied to the keratin fibers under occlusive conditions as described above.

The process for permanent deformation of keratin fibers according to the present invention may be performed without any step of oxidizing the keratin fibers. Therefore, the time required for the process according to the present invention can be shorter than that for a conventional process which needs an oxidizing step. Furthermore, damage to the keratin fibers by the oxidizing step can be avoided.

The keratin fibers may be rinsed after the step of applying the composition onto the keratin fibers and/or after the step of heating the keratin fibers.

If necessary, the composition may be applied to keratin fibers before applying mechanical tension to the keratin fibers.

(Agent and Kit)

The present invention can be performed by using an agent for one-step permanent deformation of keratin fibers to be heated in an occlusive space, comprising one or several sources of ions of formula:

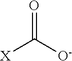

wherein

X is a group selected from the group consisting of O⁻, OH, NH₂, O—OH, and O—COO⁻.

The agent does not need to be used in combination with an oxidizing agent which is used in a conventional permanent deformation of keratin fibers. Therefore, the agent can be used in one step, whereas two steps (reducing step and oxidizing step) are necessary in the conventional permanent deformation of keratin fibers.

The agent may have the same technical features as those of the composition described above.

The present invention can be performed by using a kit for permanent deformation of keratin fibers, comprising:
a device comprising at least one reshaping means to provide the keratin fibers
with mechanical tension,
at least one coating means to form an occlusive space, and
at least one heater to heat the keratin fibers in the occlusive space;

and

a composition comprising one or several sources of ions of formula:

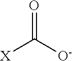

wherein

X is a group selected from the group consisting of O⁻, OH, NH₂, O—OH, and O—COO⁻.

The reshaping means, the coating means, and the heater, as well as the composition in the kit, may be the same as those described above.

### EXAMPLES

The present invention will be described in more detail by way of examples, which however should not be construed as limiting the scope of the present invention.

Compositions 1-5

The following compositions were prepared (active ingredients in wt %). The concentration of the carbonate salt or the bicarbonate salt was fixed at 0.8 M.

Composition 1

| Components | Amount (wt %) |
|---|---|
| Sodium bicarbonate | 6.7 |
| Water | qsp 100 |
| | pH 8 |

Composition 2

| Components | Amount (wt %) |
|---|---|
| Ammonium bicarbonate | 6.3 |
| Water | qsp 100 |
| | pH 8 |

Composition 3

| Components | Amount (wt %) |
|---|---|
| Tetraethylammonium bicarbonate | 15.3 |
| Water | qsp 100 |
| | pH 8.8 |

Composition 4

| Components | Amount (wt %) |
|---|---|
| Sodium carbonate | 8.4 |
| HCl | qsp pH 8 |
| Water | qsp 100 |

Composition 5

| Components | Amount (wt %) |
|---|---|
| Guanidine carbonate | 7.2 |
| HCl | qsp pH 8 |
| Water | qsp 100 |

### Examples 1-5 and Comparative Examples 1-5

### Example 1

Composition 1 was applied for 15 minutes on a 1 g Japanese hair swatch previously wrapped on a 1.7 cm perm-roller. Then the perm-roller was covered by a plastic film and plugged on a Digital Perm Machine (Oohiro, model ODIS-2). After the heating process at 90° C. for 15 minutes, the hair was rinsed and dried.

### Comparative Example 1

Composition 1 was applied for 15 minutes on a 1 g Japanese hair swatch previously wrapped on a 1.7 cm perm-roller. After the pausing time, the hair was rinsed. Then, the hair was removed from the perm-roller, rinsed again and dried.

### Example 2

Composition 2 was applied for 15 minutes on a 1 g Japanese hair swatch previously wrapped on a 1.7 cm perm-roller. Then the perm-roller was covered by a plastic film and plugged on a Digital Perm Machine (Oohiro, model ODIS-2). After the heating process at 90° C. for 15 minutes, the hair was rinsed and dried.

### Comparative Example 2

Composition 2 was applied for 15 minutes on a 1 g Japanese hair swatch previously wrapped on a 1.7 cm perm-roller. After the pausing time, the hair was rinsed. Then, the hair was removed from the perm-roller, rinsed again and dried.

### Example 3

Composition 3 was applied for 15 minutes on a 1 g Japanese hair swatch previously wrapped on a 1.7 cm perm-roller. Then the perm-roller was covered by a plastic film and plugged on a Digital Perm Machine (Oohiro, model ODIS-2). After the heating process at 90° C. for 15 minutes, the hair was rinsed and dried.

### Comparative Example 3

Composition 3 was applied for 15 minutes on a 1 g Japanese hair swatch previously wrapped on a 1.7 cm perm-roller. After the pausing time, the hair was rinsed. Then, the hair was removed from the perm-roller, rinsed again and dried.

### Example 4

Composition 4 was applied for 15 minutes on a 1 g Japanese hair swatch previously wrapped on a 1.7 cm perm-roller. Then the perm-roller was recovered by a plastic film and plugged on a Digital Perm Machine (Oohiro, model ODIS-2). After the heating process at 90° C. for 15 minutes, the hair was rinsed and dried.

### Comparative Example 4

Composition 4 was applied for 15 minutes on a 1 g Japanese hair swatch previously wrapped on a 1.7 cm perm-roller. After the pausing time, the hair was rinsed. Then, the hair was removed from the perm-roller, rinsed again and dried.

### Example 5

Composition 5 was applied for 15 minutes on a 1 g Japanese hair swatch previously wrapped on a 1.7 cm perm-roller. Then the perm-roller was covered by a plastic film and plugged on a Digital Perm Machine (Oohiro, model ODIS-2). After the heating process at 90° C. for 15 minutes, the hair was rinsed and dried.

### Comparative Example 5

Composition 5 was applied for 15 minutes on a 1 g Japanese hair swatch previously wrapped on a 1.7 cm perm-roller. After the pausing time, the hair was rinsed. Then, the hair was removed from the perm-roller, rinsed again and dried.

A curl retention test was performed on the modified hair swatches described in Examples 1-5 and Comparative Examples 1-5. For that purpose, the hair swatches were kept under straight constraints for 5 hours at 40° C. and under a 100% relative humidity. The curl long-lastingness was evaluated by comparing artificial shape before and after this curl retention test. The results are shown in Table 1.

TABLE 1

| | | Heating | Curl | Curl |
|---|---|---|---|---|
| | Reductive step | Process | Efficiency | Retention |
| Example 1 | Composition 1 | 90° C. | ++ | + |
| Comp. | Composition 1 | − | −− | −− |
| Example 1 | | | | |
| Example 2 | Composition 2 | 90° C. | ++ | + |
| Comp. | Composition 2 | − | −− | −− |
| Example 2 | | | | |
| Example 3 | Composition 3 | 90° C. | ++ | + |
| Comp. | Composition 3 | − | −− | −− |
| Example 3 | | | | |
| Example 4 | Composition 4 | 90° C. | ++ | + |
| Comp. | Composition 4 | − | −− | −− |
| Example 4 | | | | |
| Example 5 | Composition 5 | 90° C. | ++ | + |
| Comp. | Composition 5 | − | −− | −− |
| Example 5 | | | | |

++: very good curl efficiency
+: good curl efficiency
−: low curl efficiency
−−: very weak curl efficiency

Table 1 shows that the present inventions can provide better curl efficiency and curl retention effects.

1. A process for permanent deformation of a keratin fiber comprising:
providing the keratin fibers with mechanical tension for the deformation;
applying onto the keratin fibers a composition comprising at least one source of ions of formula: 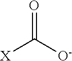
wherein
X is selected from the group consisting of O⁻, OH, NH₂, O—OH, and O—COO⁻;
placing the keratin fibers in an occlusive space; and
heating the keratin fibers.

2. The process according to claim 1, further comprising rinsing the keratin fibers after applying the composition onto the keratin fibers and/or after heating the keratin fibers.

3. The process according to claim 1, wherein the occlusive space is formed by at least one coating apparatus.

4. The process according to claim 3, wherein the coating apparatus is rigid or flexible.

5. The process according to claim 3, wherein the coating apparatus comprises at least one member selected from the group consisting of a film and a sheet.

6. The process according to claim 1, wherein the mechanical tension is provided by at least one reshaping apparatus selected from the group consisting of a curler, a roller, a plate and an iron.

7. The process according to claim 1, wherein the keratin fibers are heated at 50° C. to 250° C. during the heating of the keratin fibers.

8. The process according to claim 1, wherein the keratin fibers are heated by at least one heater providing at least one source selected from the group consisting of hot air, hot steam, high frequency induction heating, microwave heating, infra-red ray irradiation, laser, and flash lamp irradiation.

9. The process according to claim 8, wherein the coating apparatus and/or the reshaping apparatus comprise(s) the heater.

10. The process according to claim 1, wherein the composition comprises the sources of ions in a total amount of 0.1 to 40% by weight relative to the total weight of the composition.

11. The process according to claim 1, wherein the pH of the composition ranges from 6 to 11.

12. The process according to claim 1, wherein the source of ions is selected from the group consisting of carbonate salts and hydrogencarbonate salts.

13. The process according to claim 1, wherein a step of oxidizing the keratin fibers is not included.

14. An agent for one-step permanent deformation of keratin fibers, comprising at least one source of ions of formula: 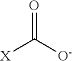
wherein
X is selected from the group consisting of O⁻, OH, NH₂, O—OH, and O—COO⁻.

15. A kit for permanent deformation of keratin fibers, comprising:
a device comprising
at least one reshaping apparatus to provide the keratin fibers with mechanical tension,
at least one coating apparatus to form an occlusive space, and
at least one heater to heat the keratin fibers in the occlusive space;
and
a composition comprising at least one source of ions of formula: 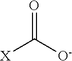
wherein
X is selected from the group consisting of O⁻, OH, NH₂, O—OH, and O—COO⁻.

16. (canceled)

17. The process of claim 1, wherein the ions are selected from the group consisting of carbonate ions, hydrogencarbonate ions, carbamate ions and peroxycarbonate ions.

18. The agent of claim 14, wherein the ions are selected from the group consisting of carbonate ions, hydrogencarbonate ions, carbamate ions and peroxycarbonate ions.

19. The kit of claim 15, wherein the ions are selected from the group consisting of carbonate ions, hydrogencarbonate ions, carbamate ions and peroxycarbonate ions.
